(19) United States
(12) Plant Patent Application Publication
(10) Pub. No.: US 2002/0199227 P1
(43) Pub. Date: Dec. 26, 2002

(54) Chrysanthemum plant named 'GEDI ONE FRI'

(76) Inventor: Dirk Pieters, Staden (BE)

Correspondence Address: KNOBBE MARTENS OLSON & BEAR LLP, 2040 MAIN STREET, FOURTEENTH FLOOR, IRVINE, CA, 92614, US

(21) Appl. No.: 09/873,957

(22) Filed: Jun. 4, 2001

(51) Int. Cl. A01H 5/00

(52) U.S. Cl. PLT/288

(57) ABSTRACT

A new and distinct Chrysanthemum plant cultivar characterized by large decorative type inflorescences, consistent flowering response to short days, blooming consistently after 49 days of short day length, free branching habit, and a consistent natural season flowering habit during the first two weeks of September.

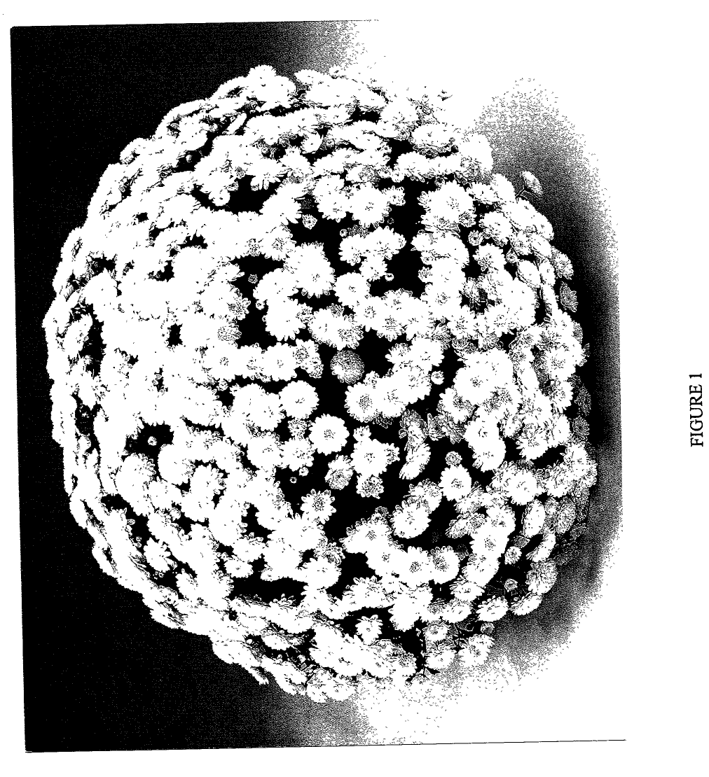

### BACKGROUND OF THE INVENTION

The present invention relates to a new and distinct cultivar of Chrysanthemum plant, botanically known as Dendranthema morifolium and hereinafter referred to by the cultivar name ‘GEDI ONE FRI.’

The new cultivar is a product of hybridization of the female parent ‘GEDI WO’ (U.S. Plant patent application Ser. No. 09/245,234) and the male parent ‘Speedo’ (unpatented.) The new cultivar was discovered and selected by the inventor in October 1997.

Asexual reproduction of the new cultivar by apical tip cutting and meristem tissue culture was performed in Oxnard, Calif. and has shown that the unique features of this new Chrysanthemum cultivar are stable and reproduced true to type on successive generations.

### SUMMARY OF THE INVENTION

The cultivar ‘GEDI ONE FRI’ has not been observed under all possible environmental conditions. The phenotype may vary somewhat with variations in environment such as temperature, daylength, and light intensity, without, however, any variance in genotype.

The following traits have been repeatedly observed and are determined to be the unique characteristics of ‘GEDI ONE FRI.’ These characteristics in combination distinguish ‘GEDI ONE FRI’ as a new and distinct Chrysanthemum cultivar:

1. Large decorative type inflorescence;

2. Consistent flowering response to short days, blooming consistently after 49 days of short day length;

3. Free branching habit; and

4. Consistent natural season flowering habit during the first two weeks of September.

Plants of the new cultivar are similar to plants of the parent cultivars, ‘Gedi WO’ and ‘Speedo’ in most horticultural characteristics, however plants of the new cultivar differed from plants of the parent cultivars primarily in bloom characteristics. In comparisons of plants of the new cultivar with the parent cultivars the following characteristics differed:

1. The new cultivar has a wider diameter flower than cultivar ‘Gedi WO;’

2. The new cultivar has many more ray florets than cultivar ‘Gedi WO;’

3. The new cultivar naturally blooms three weeks earlier than the cultivar ‘Gedi WO;’

4. The new cultivar has fully decorative flowers, while the cultivar ‘Gedi WO’ has daisy type flowers;

5. The new cultivar has a wider diameter flower than the cultivar ‘Speedo;’

6. The new cultivar has many more ray florets than the cultivar ‘Speedo;’ and

7. The new cultivar naturally blooms three weeks earlier than the cultivar ‘Speedo.’

### BRIEF DESCRIPTON OF THE INVENTION

The photograph shows a plant of the new cultivar which was grown in a 2-gallon container. A single cutting was grown in the container, and the plant was grown in outdoor field conditions.

### DETAILED BOTANICAL DESCRIPTION

In the following description, color references are made to The Royal Horticultural Society Colour Chart except where general terms of ordinary dictionary significance are used. The following observations and measurements describe plants grown in a commercial field in Oxnard, Calif. in the month of September 2000. The growing conditions range from 60° to 65° F. at night to 70° to 85° F. during the day. Measurements and numerical values represent averages of typical flowering types.

Botanical classification: Dendranthema morifolium cultivar ‘GEDI ONE FRI’

Commercial classification: Garden-type Chrysanthemum

Parentage: Hybridization of female parent ‘Gedi WO’ and male parent ‘Speedo’

### Propagation

Time to Rooting: 7 to 14 days at approximately 21° C.

Rooting habit: Fine, fibrous

### Plant

Growth Habit: Mounding herbaceous perennial

Height: Approximately 28 cm

Growth rate: Moderate

Branching Characteristics: Free Branching

### Foliage

Leaf:

Arrangement.—Alternate single.

Average length.—Approximately 7.2 cm.

Average width.—Approximately 3.7 cm.

Shape of blade.—Ovate.

Apex.—Acuminate.

Base.—Attenuate.

Attachment.—Stalked.

Margin.—Serrate.

Texture of top surface.—Lightly pubescent.

Texture of bottom surface.—Lightly pubescent.

Color.—young foliage upper side: Near 137A young foliage underside: Near 137B mature foliage upper side: Near 137A mature foliage underside: Near 137B venation upper side: Near 138C venation under side: Near 137C

Petiole:

Average length.—Approximately 2.3 cm.

### Bloom

Inflorescence:

Flowering habit.—Induced by darkness period greater than 13.5 hours, approximately 49 days of appropriate day length required to induce and develop blooms.

Inflorescence form.—Decorative.

Natural flowering season.—Approximately the last two weeks in September.

Number of inflorescences per stem.—Approximately 11.

Inflorescence diameter.—Approximately 6 cm.

Inflorescence depth.—1.5 cm.

Inflorescence longevity on plant.—Approximately 22-26 days

Persistence.—Persistent.

Ray Florets:

Appearance.—Matte.

Texture.—Smooth.

Average number per flower.—250.

Shape.—Oblanceolate.

Margin.—Entire.

Apex.—Obtuse.

Length.—Approximately 3.5 cm.

Width.—Approximately 0.8 cm.

Color.—upper surface at first opening: Near 18B upper surface at maturity: Near 158D upper surface at fading: Near 155B under surface at first opening: Near 18C under surface at maturity: Near 158D under surface at fading: Near 155B

Disc florets:

Appearance.—Shiny.

Texture.—Smooth.

Average number per flower.—Approximately 40.

Shape.—Cylindric.

Apex.—Obtuse.

Average length.—Approximately 1 cm.

Average width.—Approximately 0.2 cm.

Color.—at first opening: Near 2A at maturity: Near 16B at fading: Near 17D

Peduncle:

Length.—at terminal end (shortest): Approximately 5 cm. at lateral end (longest): Approximately 9 cm.

Strength.—Moderate.

Color.—Near 137C.

Habit.—Erect.

Inflorescence bud:

Length.—Approximately 1 cm.

Diameter.—Approximately 1 cm.

Form.—Globular.

Color.—Near 16C.

Sepals:

Appearance.—Matte.

Texture.—Lightly pubescent.

Number.—Approximately 40 to 50.

Shape.—Ovate.

Margin.—Entire.

Apex.—Acute.

Color.—upper side: Near 137A under side: Near 137A

### Reproductive Organs

Ray Florets:

Pistillate.—Number of pistils per flower: 1 Stigma shape: 2 branched Stigma color: Near 16D Style color: Near 16D Style length: 0.6 cm

Disc Florets:

Pistillate.—Number of pistils per flower: 1 Stigma shape: Cylindric Style length: 0.5 cm

Staminate.—Number of stamens per flower: Approximately 5 Anther shape: Tubular Anther color: Near 16A Pollen color: Near 17B

### ‘GEDI ONE FRI’: Table of R.H.S. Color Codes

| | | | |
|---|---|---|---|
| FOLIAGE | Leaf | young foliage upper surface | Near 137A |
| | | young foliage under surface | Near 137B |
| | | mature foliage upper surface | Near 137A |
| | | mature foliage under surface | Near 137B |
| | | venation upper surface | Near 138C |
| | | venation under surface | Near 137D |
| BLOOM | Ray Florets | upper surface at first opening | Near 18B |
| | | upper surface at maturity | Near 158D |
| | | upper surface at fading | Near 155B |
| | | under surface at first opening | Near 18C |
| | | under surface at maturity | Near 158D |
| | | under surface at fading | Near 155B |
| | | Pistillate: Stigma | Near 16D |
| | | Pistillate: Style | Near 16D |
| | Disc Florets | at first opening | Near 2A |
| | | at maturity | Near 16B |
| | | at fading | Near 17D |
| | | Staminate: Anther | Near 16A |
| | | Staminate: Pollen | Near 17B |
| | Peduncle | | Near 137C |
| | Inflorescence | | Near 16C |
| | Bud | | |
| | Sepals | upper surface | Near 137A |
| | | under surface | Near 137A |

What is claimed is:

1. A new and distinct cultivar of Chrysanthemum plant named ‘GEDI ONE FRI’ as herein illustrated and described.
